(19) United States
(12) Patent Application Publication
(10) Pub. No.: US 2013/0102033 A1
(43) Pub. Date: Apr. 25, 2013

(54) PRODUCTION METHOD OF EXTRACELLULAR POLYSACCHAROPEPTIDES BY TRAMETES VERSICOLOR LH1 IMMOBILIZATION USING LIQUID-SURFACE COMPLEX CARRIERS IN SUBMERGED FERMENTATION

(71) Applicants: Tai-Hao HSU, Changhua County (TW); Fang-Yi Lin, Changhua County (TW)

(72) Inventors: Tai-Hao HSU, Changhua County (TW); Fang-Yi Lin, Changhua County (TW)

(21) Appl. No.: 13/276,944

(22) Filed: Oct. 19, 2011

(51) Int. Cl. C12P 21/06

(52) U.S. Cl. 435/71.1

(57) ABSTRACT

The present invention relates to a culture method for enhancing the production of the extracellular polysaccharopeptides (EPS) by submerged fermentations with a mycelium immobilization by a local Trametes versicolor strain LH1 and significantly increasing the production of polysaccharopeptides with immunomodulatory activity of Trametes versicolor LH1 through mycelium immobilization by liquid-surface complex carrier with gelling material at optimal condition. This method is beneficial to mycelium re-use in fed-batch submerged fermentation by providing a better micro-environment and simplifying downstream processes of isolating and recycling the EPS from broth.

### FIELD OF THE INVENTION

The present invention relates to a technical area of medical polysaccharopeptides, in particular to a production method of extracellular polysaccharopeptides (EPS) by using trametes versicolor to promote the production yield.

### BACKGROUND OF THE INVENTION

Trametes versicolor (syn. Coriolus versicolor) is a white strain parasitic on rotten wood and grown in places all over the world, and it is a widely used medical fungus (Wasser, & Weis, 1999). The fruiting body of trametes versicolor grows slowly and has a high price, and the trametes versicolor polysaccharopeptides produced by a liquid culture method is found to have the immunomodulatory and anti-tumor biological activities (Cui, & Chisti, 2003). The trametes versicolor polysaccharopeptides can be isolated by different trametes versicolor strains such as CM-101(ATCC 20547) or Cov-1 isolation (Kobayashi, Matsunaga, & Fujii, 1993; Cui, & Chisti, 2003; Moradali, Mostafavi, Ghods, & Hediaroude, 2007). In vitro, in vivo, and clinical experiments show that polysaccharides in the polysaccharopeptides have immunomodulatory activities by α(1→4) and β(1→3) bonds (Hsieh, Wu, Park, & Wu, 2006). The polysaccharopeptides produced by trametes versicolor strains Cov-1 can increase the secretion of immune cells of interleukin IL-1β and IL-6 and induce the apoptosis of human HL-60 lymphoma cells (Hsieh, Kunicki, Darzynkiewicz, & Wu, 2002).

In vivo animal experiments show that macrophages isolated from mice fed with polysaccharopeptides contain reactive nitrogen intermediates (RNI), superoxide anions and tumor necrosis factor-γ (TNF-γ) (Liu, Ng, Sze, & Tsui, 1993). The polysaccharopeptides of the trametes versicolor can activate the macrophages, stimulate the secretion of cytokines TNF-γ, interferon-γ (IFN-γ) and interleukin-1β (IL-1β), and these cytokines are provided for againsting the cell proliferation and causing the apoptosis and differentiation. The polysaccharopeptides of the trametes versicolor providing a non-specific defense of the immune system plus the patient's immune system can produce an anti-tumor effect (Ng, 1998).

In the culture of a general fungal submerged fermentation tank, the growth of strains and the production of extracellular polysaccharopeptides will be affected by the culture conditions and environments and the composition of the culture medium. Boletus spp is used as an example, and the cultured strain and the extracellular polysaccharopeptides are affected by factors including temperature, stirring speed and initial pH value (Wang, & Lu, 2005). Different trametes versicolor strains Wr-74 and ATCC-20545 add milk serum into the culture medium to affect the of the production yield of the strains and extracellular polysaccharopeptides (Cui, Goh, Archer, & Singh, 2007) and also affect the anti-tumor activity of the polysaccharopeptides of the trametes versicolor during the culture period (Lee et al., 2006). In the conventional liquid culture process of the trametes versicolor, the production yield of the intracellular polysaccharopeptides or extracellular polysaccharopeptides is not high, or the conventional process has not adopted the novel immobilization technique to produce the extracellular polysaccharopeptides (EPS) to enhance the production yield of the polysaccharopeptides of the trametes versicolor under the same culture conditions.

### REFERENCES

1. Cui, J. and Chisti, Y. (2003) Polysaccharopeptides of Coriolus versicolor: physiological activity, uses and production. Biotechnology Advances, 21(2), 109-122.
2. Cui, J., Goh, K. K., Archer, R. and Singh, H. (2007) Characterisation and bioactivity of protein-bound polysaccharides from submerged-culture fermentation of Coriolus versicolor Wr-74 and ATCC-20545 strains. Journal of Industrial Microbiology & Biotechnology, 34(5), 393-402.
3. Hsieh, T. C., Kunicki, J., Darzynkiewicz, Z. and Wu, J. M. (2002) Effects of extracts of Coriolus versicolor (I'm-Yunity) on cell-cycle progression and expression of interleukins-1 beta-6, and -8 in promyelocytic HL-60 leukemic cells and mitogenically stimulated and nonstimulated human lymphocytes. Journal of Alternative and Complementary Medicine, 8(5), 591-602.
4. Hsieh, T. C., Wu, P., Park, S. and Wu, J. M. (2006) Induction of cell cycle changes and modulation of apoptogenic/anti-apoptotic and extracellular signaling regulatory protein expression by water extracts of I'm-Yunity™ (PSP). BMC Complementary and Alternative Medicine, 6, 30.
5. Kobayashi, H., Matsunaga, K. and Fujii, M. (1993) PSK as a chemopreventive agent. Cancer Epidemiology, Biomarkers Prevention, 2(3), 271-276.
6. Lee, C. L., Yang, X. and Wan, M. F. (2006) The culture duration affects the immunomodulatory and anticancer effect of polysaccharopeptide derived from Coriolus versicolor. Enzyme and Microbial Technology, 38(1-2), 14-21.
7. Lin, F. Y., Lai, Y. K., Yu, H. C., Chen, N. Y., Chang, C. Y., Lo, H. C. and Hsu, T. H. (2008) Effects of Lycium barbarum extract on production and immunomodulatory activity of the extracellular polysaccharopeptides from submerged fermentation culture of Coriolus versicolor. Food Chemistry. 110(2), 446-453.
8. Liu, W. K., Ng, T. B., Sze, S. F. and Tsui, K. W. (1993) Activation of peritoneal macrophages by polysaccharopeptide from the mushroom, Coriolus versicolor. Immunopharmacology, 26(2), 139-146.
9. Moradali, M. F., Mostafavi, H., Ghods, S. and Hedjaroude, G. A. (2007) Immunomodulating and anticancer agents in the realm of macromycetes fungi (macrofungi). International Immunopharmacology, 7 (6), 701-724.
10. Ng, T. B. (1998) A review of research on the protein-bound polysaccharide (polysaccharopeptide, PSP) from the mushroom Coriolus versicolor (Basidiomycetes: Polyporaceae). General Pharmacology, 30(1), 1-4.
11. Wang, Y. X. and Lu, Z. X. (2005) Optimization of processing parameters for the mycelial growth and extracellular polysaccharide production by Boletus spp. ACCC 50328. Process Biochemistry, 40(3-4), 1043-1051.
12. Wasser, S. P. and Weis, A. L. (1999) Therapeutic effects of substances occurring in higher Basidiomycetes mushrooms: a modern perspective. Critical Reviews in Immunology, 19(1), 65-96.

The references mentioned herein are hereby incorporated by reference.

### SUMMARY OF THE INVENTION

The inventor of the present invention collected fruit bodies of various trametes versicolors from Nantou Mountain Area of Taiwan. After the fruit bodies are isolated and cultured, it is found that one of the trametes versicolor strains (LH-1) grows quickly with a high production yield, and this trametes versicolor was identified by the Bioresource Collection and Research Center of Food Industry Research and Development Institute of Republic of China (Taiwan) as Trametes versicolor (L.) Lloyd (refer to the attached strain identification report), and studies show that it can produce new extracellular polysaccharopeptides (EPS) (or ePSP-Cv), and the conditions used for the culture medium provide a high production formation rate and a better immunomodulatory activity of the cytokine secretion (Lin et al., 2008). To further enhance the production of the extracellular polysaccharopeptides (EPS), the present invention develops a novel biological immobilization method that uses a liquid-surface complex carrier to immobilize and combine a submerged membrane reactor system to immobilize the trametes versicolor strains, and a process carried out in a 5-liter fermentation tank provides evidences. Compared with those without being processed by the mycelium immobilization, the production of the extracellular polysaccharopeptides (EPS) is improved significantly by 2.13-folds (increased from 3.94 g/L to 8.39 g/L), and the production yield of the intracellular polysaccharopeptides is improved by 7.15-folds (increased from 9.27 mg/g to 66.27 mg/g), indicating a substantial improvement. The extracellular polysaccharopeptides (EPS) of the trametes versicolor has effects on the Immunomodulatory and anti-tumor activity, not only serving as a raw material of a new medicine, but also serving as an additive for health food and cosmetics. Obviously, the present invention has a high industrial value.

### BRIEF DESCRIPTION OF THE DRAWINGS

FIG. 1 shows the effect of various types of supports on extracellular polysaccharopeptides (EPS) and intracellular polysaccharopeptides of trametes versicolor after a mycelium immobilization takes place and their biomass formation;

FIG. 2 shows the effect of various types of gels of complex polyacrylonitrile hollow microspheres on extracellular polysaccharopeptides (EPS) and intracellular polysaccharopeptides of trametes versicolor after a mycelium immobilization takes place and their biomass formation;

FIG. 3 shows the effect of agar at various concentrations of complex polyacrylonitrile hollow microspheres on extracellular polysaccharopeptides (EPS) and intracellular polysaccharopeptides of trametes versicolor after a mycelium immobilization takes place and their biomass formation;

FIG. 4 shows the effect of various inoculative quantities of trametes versicolor strains on agar of complex polyacrylonitrile hollow microspheres on extracellular polysaccharopeptides (EPS) and intracellular polysaccharopeptides of trametes versicolor after a mycelium immobilization takes place and their biomass formation;

FIG. 5 shows a change of dissolved oxygen and pH values of extracellular polysaccharopeptides (EPS) in a batch-culture production procedure of agar complex polyacrylonitrile hollow microspheres of immobilized trametes versicolor strains and a culture medium; and

FIG. 6 shows a change of dissolved oxygen and pH values of extracellular polysaccharopeptides (EPS) in a continuous fed-batch culture production procedure of agar complex polyacrylonitrile hollow microspheres of immobilized trametes versicolor strains and a culture medium.

### DESCRIPTION OF THE PREFERRED EMBODIMENTS

Trametes versicolor (Trametes versicolor LH1)

A sample of the trametes versicolor is deposited at Bioresource Collection and Research Center of Food Industry Research and Development Institute of Republic of China (Taiwan) with a registration number of BCRC 930112.

Liquid culture of trametes versicolor, extracellular polysaccharopeptides and intracellular polysaccharopeptides

The foregoing deposited trametes versicolor is scraped and inoculated on a culture medium. At a temperature of 25° C., the culture medium with the trametes versicolor strain is shaken and cultured in a constant-temperature shaking incubator at an oscillation rate of 150 rpm for 5 days to produce a stain.

Formula of Culture Medium

| Ingredient | Concentration (%) |
|---|---|
| Glucose | 4 |
| Peptone | 0.3 |
| Potassium dihydrogen | 0.15 |
| phosphate (KH₂PO₄) | |
| Magnesium sulfate | 0.15 |
| (MgSO₄ 7H₂O) | |

The formula of the culture medium in accordance with a preferred embodiment is given below:

The aforementioned culture medium with an inoculation quantity of 10% is placed and cultured in a 500 mL conical flask (at a temperature of 25° C. and a stirring speed of 150 rpm, and with a culture medium quantity of 100 mL). The fermentation medium obtained after 7 days of culture is centrifuged at 6000 rpm for 30 minutes to obtain a strain and a supernatant. The supernatant is put into a 95% alcohol with four times of the quantity and precipitated for 24 hours and then centrifuged, and the precipitate contains a composite of polysaccharides and proteins and the precipitate polysaccharopeptides are extracellular polysaccharopeptides (EPS) according to the “phenol-sulphuric acid method” by Dubois, et al (1956) and the AOAC method (1995).

The strains in the immobilized support is washed by sterile water for three times, and a non-dissolved support material is extracted directly by heat in a high-pressure steam sterilizer for 30 minutes (at 121° C.), and the dissolved support material is added with the same amount of distilled water, and a hot water bath is provided for dissolving a support portion containing the mycelium immobilization support, and strains are prepared by a centrifuge. The centrifuged strains are frozen and dried, and 1 g of the strains is assed into 10 mL of distilled water, and extracted in an autoclave for 30 minutes (at 121° C.), and then exhausted and filtered. The filtered solution is collected, and the strains are extracted by hot water to produce an extracted solution. 1 mL of the extracted solution is added into 4 mL of 95% ethanol, and set still at 4° C. for 24 hours, and then centrifuged at 7000 rpm for 10 minutes to remove the supernatant. 1 mL of R.O. water is added, and the solution is put into a freezer at −18° C. and frozen and dried, the precipitated polysaccharopeptides is the intracellular polysaccharopeptides (IPS).

Strain Biomass Measurement

The fermentation medium is centrifuged at 6000 rpm for 30 minutes to obtain the strains and supernatant. The strains is added into 10 ml of deionized water for a suspension cleaning and centrifuged at 6000 rpm for 30 minutes, and the centrifuge is repeated for three times. The obtained strains are frozen, dried, and weighed, and the products are biomass strains. In the mycelium immobilization experiment, the immobilized support material is dried and weighed till a constant weight is obtained before the strains are inoculated. After the experiment is finished, the support containing the strains is frozen and dried, and the dry weight deducting the original weight of the support material is the biomass of the strains.

Statistical Analysis

Experiment data of the present invention are analyzed by the Statistical Analysis System 6.0 software, and ANOVA is used for the variation analysis, and the Duncan's multiple range test is used for comparing the significant difference of the means (p<0.05).

### Embodiment 1

The first embodiment shows the effect of various types of supports on extracellular polysaccharopeptides (EPS) and intracellular polysaccharopeptides of trametes versicolor after a mycelium immobilization takes place and their biomass formation.

The present invention mainly uses a 5-liter fermentation tank for the related experiments, and the preparation of complex carrier materials will be described below. In the design of apparatuses, a three-state culture system is developed: (1) Gas State Layer: This layer is a liquid-surface immobilized system primarily made of heat-resisting hollow microsphere of polycarbonate, polypropylene and polyacrylonitrile, and a mycelium immobilization is formed after the gel is combined; (2) Liquid State Layer: This layer is a strain containing carrier prepared by the culture medium and gone through the complex immobilization; and (3) Solid State Layer: This layer is a submerged membrane module placed at the bottom of the fermentation tank for filtering possible remaining portions of strains.

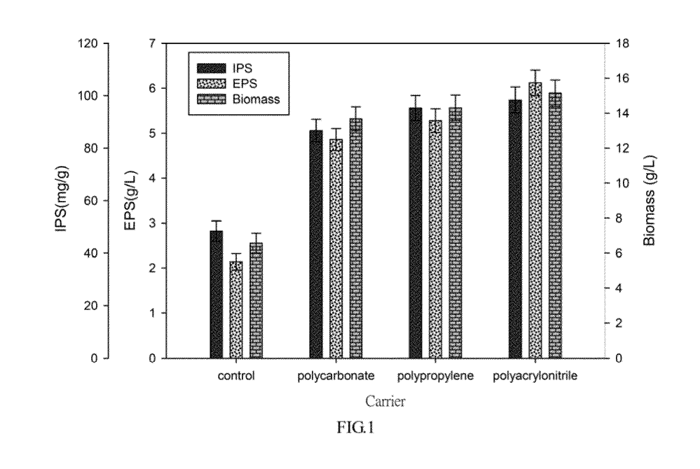

Three materials including the heat-resisting polycarbonate, polypropylene, and polyacrylonitrile hollow microspheres are used as the supports of the mycelium immobilization. The former two types of materials with a constant weight are placed into the 5-liter fermentation tank, and the concentration of the later two types of materials is 5% and in a solution form filled into the 5-liter fermentation tank. The 5-liter fermentation tank is placed into a high-pressure steam sterilizer and sterilized by steam at 121° C. for 30 minutes. Three liters of solution containing 5% strains with a concentration (10 g/L) is added into the culture medium fed-in fermentation tank, and cultured at 25° C. for 4 days in the ventilation of 1 VVM. By the aforementioned analysis method, the extracellular polysaccharopeptides (EPS), intracellular polysaccharopeptides (IPS) and biomass are analyzed, and the analysis results are shown in FIG. 1. Among the three immobilized support materials, the extracellular polysaccharopeptides (EPS), intracellular polysaccharopeptides (IPS) and biomass produced by the liquid culture of the trametes versicolor, the polyacrylonitrile hollow microsphere is the best, and the polypropylene hollow microsphere is the second best. Compared with the control group of non-immobilized ionized strains, these immobilized support materials have much better production yield.

### Embodiment 2

The second embodiment shows the effect of various types of gels of complex polyacrylonitrile hollow microspheres on extracellular polysaccharopeptides (EPS) and intracellular polysaccharopeptides of trametes versicolor after a mycelium immobilization takes place and their biomass formation.

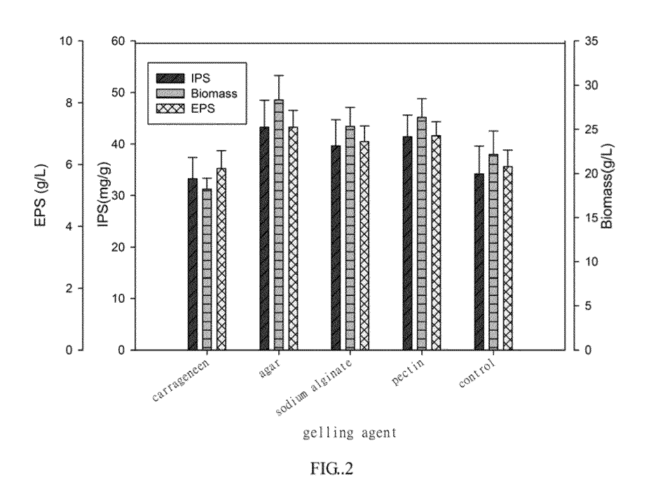

According to the results obtained from the first embodiment, the polyacrylonitrile hollow microsphere is pre-processed by four gels (including carrageenan, agar or agarose, alginate and pectin) of the concentration of 2%. In other words, the polyacrylonitrile hollow microspheres are placed into the foregoing gel solution for 1 minute to form an immobilized support of the gel of a complex polyacrylonitrile hollow microsphere. The main effect is to enhance the mycelium immobilization by a simple method, and the related results are shown in FIG. 2. Among the four mycelium immobilization gels of the complex polyacrylonitrile hollow microspheres, the extracellular polysaccharopeptides (EPS), intracellular polysaccharopeptides (IPS) and biomass produced by the liquid culture of the trametes versicolor, agar is the best, and pectin is the second best.

### Embodiment 3

The third preferred embodiment shows the effect of agar at various concentrations of complex polyacrylonitrile hollow microspheres on extracellular polysaccharopeptides (EPS) and intracellular polysaccharopeptides of trametes versicolor after a mycelium immobilization takes place and their biomass formation.

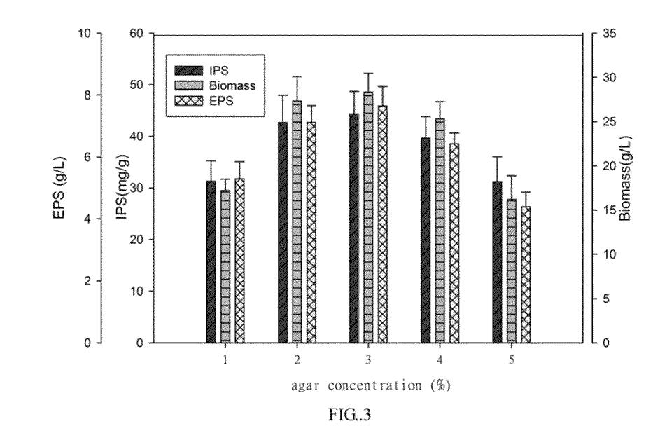

According to the conclusion obtained from the second embodiment, 5 types of agars at the concentrations of (1%, 2%, 3%, 4%, and 5%) of the complex polyacrylonitrile hollow microspheres are produced by the most suitable material, and the related experiment results are shown in FIG. 3. The results show that among the extracellular polysaccharopeptides (EPS), intracellular polysaccharopeptides (IPS) and biomass produce by the liquid-culture mycelium immobilization of the trametes versicolor, the complex polyacrylonitrile hollow microsphere of the 3% agar is the best.

### Embodiment 4

The fourth preferred embodiment shows the effect of various inoculation quantities of trametes versicolor strains on agar of complex polyacrylonitrile hollow microspheres on extracellular polysaccharopeptides (EPS) and intracellular polysaccharopeptides of trametes versicolor after a mycelium immobilization takes place and their biomass formation.

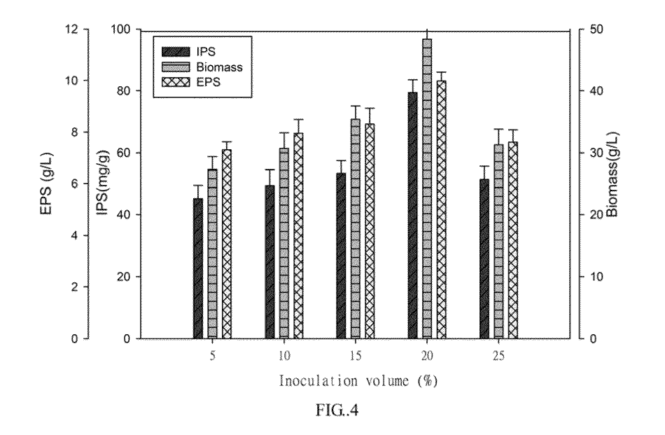

According to the conclusion obtained from the third embodiment, different inoculation amounts (5%, 10%, 15%, 20% and 25%) of the trametes versicolor are inoculated to improve the production yield of the extracellular polysaccharopeptides (EPS) and intracellular polysaccharopeptides and biomass, and the results are shown in FIG. 4. The results show that if the inoculation amount falls within a range from 5% to 20%, the extracellular polysaccharopeptides (EPS), intracellular polysaccharopeptides and biomass of the trametes versicolor increase as the inoculation amount increases.

In this embodiment, the extracellular polysaccharopeptides (EPS), intracellular polysaccharopeptides and biomass of the trametes versicolor obtained from the inoculation amount of 20% are 9.98 g/L, 79.37 mg/g and 48.36 g/L respectively. Compared with the control group of the first embodiment (without being processed by the mycelium immobilization), the extracellular polysaccharopeptides (EPS), intracellular polysaccharopeptides and biomass of the trametes versicolor without being processed by the mycelium immobilization are 3.94 g/L, 9.27 mg/g and 9.31 g/L, respectively. In other words, the preparation and processing by mycelium immobilization can increase the production yield of the extracellular polysaccharopeptides (EPS) of the trametes versicolor to 2.53-folds, the intracellular polysaccharopeptides to 8.56-folds, and the biomass to 5.19-folds.

### Embodiment 5

The fifth preferred embodiment shows a change of dissolved oxygen and pH values of extracellular polysaccharopeptides (EPS) in a batch-culture production procedure of agar complex polyacrylonitrile hollow microspheres of immobilized trametes versicolor strains and a culture medium.

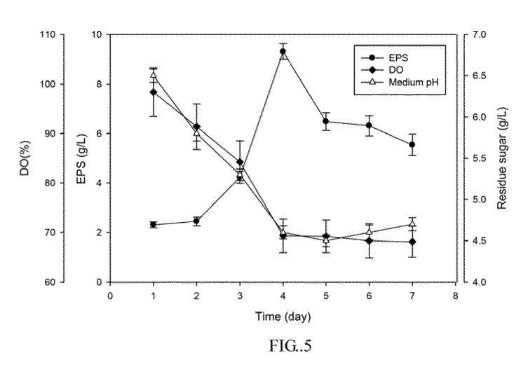

According to the conclusion obtained from the fourth embodiment, the 5-liter fermentation tank is provided for analyzing the correlation between the pH value of the culture medium pH and the dissolved oxygen (DO) of the culture medium during the 7-day production course of the extracellular polysaccharopeptides (EPS) of the trametes versicolor, and appropriate condition and feeding time for the production control have been established. Related results are shown in FIG. 5, and the results show that on the second day of the culture, the production of the extracellular polysaccharopeptides (EPS) of the trametes versicolor has a significant increase, and climbs up to the peak on the fourth day, and then starts declining thereafter. The pH value of the culture medium and the dissolved oxygen of the culture medium decrease as the production of the extracellular polysaccharopeptides (EPS) increases. When the production of the extracellular polysaccharopeptides (EPS) drops, the pH value of the culture medium and the dissolved oxygen of the culture medium remains at a stable state, indicating that the change of the pH value of the culture medium and the dissolved oxygen of the culture medium can be used for an immediate monitoring to facilitate controlling the appropriate feeding time.

In this embodiment, the extracellular polysaccharopeptides (EPS) of the trametes versicolor obtained on the fourth day of the culture has a maximum production yield of 9.31 g/L, which is 2.36-folds of the production yield of 3.94 g/L for the extracellular polysaccharopeptides (EPS) of the trametes versicolor being processed by the fermentation process of the mycelium immobilization (such as the control of the first embodiment, sufficiently indicating that the present invention can improve over the prior art.

### Embodiment 6

The sixth preferred embodiment shows a change of dissolved oxygen and pH values of extracellular polysaccharopeptides (EPS) in a continuous fed-batch culture production procedure of agar complex polyacrylonitrile hollow microspheres of immobilized trametes versicolor strains and a culture medium.

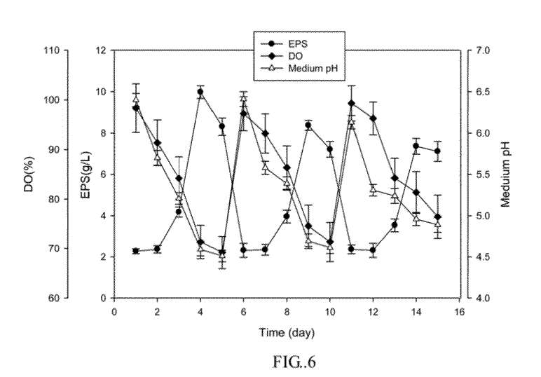

According to the conclusion obtained from the second embodiment, continuous fed-batch culture production procedure is carried out to show that the feasibility of the continuous repeated operation of the support of the mycelium immobilization. In other words, if the pH value of the culture medium and the dissolved oxygen decreases to the minimum, the fermentation medium containing the extracellular polysaccharopeptides (EPS) of the trametes versicolor flows out. After the fermentation medium is exhausted, a fresh culture medium is fed timely, and two times of feeding takes place in the 15-day fermentation process, wherein one feeding takes place on the fifth day, and the other takes place on the tenth day. Related results are shown in FIG. 6, and the results show that the peaks occur on the fourth to fifth day, the ninth to tenth day, and the fourteenth to fifteenth day of the culture of the extracellular polysaccharopeptides (EPS) of the trametes versicolor, sufficiently indicating that the method of the present invention is feasible.

What is claimed is:

1. A liquid culture method for enhancing a production of extracellular polysaccharopeptides (EPS) and intracellular polysaccharopeptides by trametes versicolor strain, characterized in that a porous substrate is applied for mycelium immobilization.

2. The culture method of claim 1, wherein the trametes versicolor strain is a trametes versicolor LH1, and a sample of the trametes versicolor strain is deposited at Bioresource Collection and Research Center of Food Industry Research and Development Institute of Republic of China (Taiwan) with a registration number of BCRC 930112.

3. The culture method of claim 1, wherein the polysaccharopeptides are products produced by performing a submerged culture of the trametes versicolor strain by a liquid culture medium, and separating by mycelium immobilization or fermentation culture solution.

4. The culture method of claim 3, wherein a support used during mycelium immobilized includes a biological material or a non-biological material with appropriate pores.

5. The culture method of claim 4, wherein the biological material or non-biological material with appropriate pores includes a hollow microsphere selected from the collection of polycarbonate, polypropylene and polyacrylonitrile.

6. The culture method of claim 5, wherein the biological material or non-biological material includes a complex of a viscous gel.

7. The culture method of claim 6, wherein the viscous gel includes a carrageenan, an agar (which is an agarose), an alginate and a pectin.

8. The culture method of claim 7, wherein the viscous gel is added with a concentration falling within a range from 1% to 5%.
